(19) United States
(12) Patent Application Publication
(10) Pub. No.: US 2016/0374838 A1
(43) Pub. Date: Dec. 29, 2016

(54) DRUG-ELUTING COATINGS ON POLY(DL-LACTIDE)-BASED SCAFFOLDS

(71) Applicant: Abbott Cardiovascular Systems Inc., Santa Clara, CA (US)

(72) Inventors: Stephen D. Pacetti, San Jose, CA (US); Alexander J. Sheehy, Redwood City, CA (US); Chad J. Abunassar, San Francisco, CA (US)

(21) Appl. No.: 14/754,479

(22) Filed: Jun. 29, 2015

(51) Int. Cl. A61F 2/915

(52) U.S. Cl. CPC A61F 2/915; A61F 2002/91558; A61F 2002/91566; A61F 2210/0004; A61F 2210/0076; A61F 2250/0067

(57) ABSTRACT

Stents including a poly(D,L-lactide)(PDLLA)-based scaffold and PDLLA based therapeutic layer are disclosed. The PDLLA based scaffold may be amorphous and may include a primer layer. Methods of applying the PDLLA-based coating to the scaffold are disclosed with solvent processing methods using a solvent blend are also disclosed. Methods of removing residual solvent from a PDLLA-base coating that also condition the scaffold are disclosed. Methods of treating restenosis that release drugs to prevent restenosis without interfering with the natural positive remodeling of a vessel are disclosed.

### BACKGROUND OF THE INVENTION

Field of the Invention

This invention relates polymeric medical devices, in particular, bioresorbable stents or scaffolds including polymer and drug coatings.

Description of the State of the Art

This invention relates to radially expandable endoprostheses that are adapted to be implanted in a bodily lumen. An “endoprosthesis” corresponds to an artificial device that is placed inside the body. A “lumen” refers to a cavity of a tubular organ such as a blood vessel. A stent is an example of such an endoprosthesis. Stents are generally cylindrically shaped devices that function to hold open and sometimes expand a segment of a blood vessel or other anatomical lumen such as urinary tracts and bile ducts. Most current stents are metallic and are permanent implants. Temporary stents exist and are often referred to as scaffolds as their lifetime in vivo is finite. Such scaffolds are intended to be bioresorbable, bioerodible, bioabsorbable or biodegradable. Stents and scaffolds are often used in the treatment of atherosclerotic stenosis in blood vessels. “Stenosis” refers to a narrowing or constriction of a bodily passage or orifice. In such treatments, stents reinforce body vessels and prevent restenosis following angioplasty in the vascular system. “Restenosis” refers to the reoccurrence of stenosis in a blood vessel or heart valve after it has been treated (as by balloon angioplasty, stenting, or valvuloplasty) with apparent success.

Stents are typically composed of a scaffold or scaffolding that includes a pattern or network of interconnecting structural elements or struts, formed from wires, tubes, or sheets of material rolled into a cylindrical shape. This scaffolding gets its name because it possibly physically holds open and, if desired, expands the wall of the passageway. Typically, stents are capable of being compressed or crimped onto a catheter so that they can be delivered to and deployed at a treatment site.

Delivery includes inserting the stent through small lumens using a catheter and transporting it to the treatment site. Deployment includes expanding the stent to a larger diameter once it is at the desired location. Mechanical intervention with stents has reduced the rate of restenosis as compared to balloon angioplasty. Yet, restenosis remains a significant problem. When restenosis does occur in the stented segment, its treatment can be challenging, as clinical options are more limited than for those lesions that were treated solely with a balloon.

Stents are generally made to withstand the structural loads, namely radial compressive forces, imposed on the stent as it supports the walls of a vessel. Therefore, a stent must possess adequate radial strength if its function is to support a vessel at an increased diameter. Radial strength, which is the ability of a stent to resist radial compressive forces, relates to a stent's radial yield strength and radial stiffness around a circumferential direction of the stent.

Radial strength, which is the ability of a stent to resist radial compressive forces, relates to a stent's radial yield strength and radial stiffness around a circumferential direction of the stent. A stent's “radial yield strength” or “radial strength” (for purposes of this application) may be understood as the compressive loading or pressure, which if exceeded, creates a yield stress condition resulting in the stent diameter not returning to its unloaded diameter, i.e., there is irrecoverable deformation of the stent. See, T. W. Duerig et al., Min Invas Ther & Allied Technol 2000: 9(3/4) 235-246. Stiffness is a measure of the elastic response of a device to an applied load and thus will reflect the effectiveness of the stent in resisting diameter loss due to vessel recoil and other mechanical events. Radial stiffness can be defined for a tubular device such as stent as the hoop force per unit length (of the device) required to elastically change its diameter. The inverse or reciprocal of radial stiffness may be referred to as the compliance. See, T. W. Duerig et al., Min Invas Ther & Allied Technol 2000: 9(3/4) 235-246.

When the radial yield strength is exceeded, the stent is expected to yield more severely and only a minimal force is required to cause major deformation. Radial strength is measured either by applying a compressive load to a stent between flat plates or by applying an inwardly-directed radial load to the stent.

Some treatments with stents require its presence for only a limited period of time. Once treatment is complete, which may include structural tissue support and/or drug delivery, it may be desirable for the stent to be removed or disappear from the treatment location. One way of having a stent disappear may be by fabricating a stent in whole or in part from materials that erode or disintegrate through exposure to conditions within the body. Stents fabricated from biodegradable, bioabsorbable, bioresorbable, and/or bioerodable materials such as bioresorbable polymers can be designed to completely erode only after the clinical need for them has ended.

Stents are used not only for mechanical intervention but also as vehicles for providing biological therapy. Biological therapy uses medicated stents to locally administer a therapeutic substance. A medicated stent may be fabricated by coating the surface of either a metallic or polymeric scaffold with a polymeric carrier that includes an active or bioactive agent or drug. Polymeric scaffolds may also serve as a carrier of an active agent or drug. An active agent or drug may also be included on a scaffold without being incorporated into a polymeric carrier.

### INCORPORATION BY REFERENCE

All publications, patents, and patent applications mentioned in this specification are herein incorporated by reference to the same extent as if each individual publication, patent, or patent application was specifically and individually indicated to be incorporated by reference, and as if each said individual publication, patent, or patent application was fully set forth, including any figures, herein.

### SUMMARY OF THE INVENTION

An embodiment of the present invention includes a stent comprising: a scaffold comprising a poly(D,L-lactide)(PDLLA)-based polymer having at least 50% L-enantiomer and at least 4% D-enantiomer, wherein the polymer is amorphous; and a therapeutic coating disposed over at least a portion of a surface of the scaffold, wherein the therapeutic layer comprises a drug mixed within a coating polymer composed of a poly(D,L-lactide) or poly(D,L-lactide-co-caprolactone).

The embodiment may include one or any combination of the following aspects: wherein the PDLLA-based polymer is selected from the group consisting of 50/50 PDLLA, 96/4 PDLLA, and a copolymer thereof; wherein the drug is selected from the group consisting of everolimus, rapamycin, novolimus, zotarolimus, and biolimus; wherein the coating is disposed over at least a portion of the abluminal surface of the scaffold only; wherein a lactide monomer content of the scaffold is 0.01 to 1 wt % of the scaffold; wherein a thickness of the coating is between 1 and 10 microns; wherein a thickness of the coating is between 10 and 20 microns.

An embodiment of the present invention includes a stent comprising: a scaffold including a first poly(D,L-lactide)(PDLLA)-based polymer; a primer layer on a surface of the scaffold, wherein the primer layer comprises a second PDLLA-based polymer and the primer layer is free of a therapeutic agent and; a therapeutic layer over the primer layer, wherein the therapeutic layer comprises a third PDLLA-based polymer and a drug, wherein the primer layer improves adhesion of the therapeutic layer to the scaffold.

The embodiment may include one or any combination of the following aspects: wherein a luminal surface of the scaffold is free of the therapeutic layer and the therapeutic layer is over an entire abluminal surface of the scaffold and part of the sidewall surfaces, wherein the primer layer is between the therapeutic layer and the scaffold surface; wherein the therapeutic layer is over at least a portion of the abluminal surface of the scaffold only, the primer layer is between the therapeutic layer and the scaffold surface, and the sidewalls and luminal surface are free of the therapeutic layer; wherein the first PDLLA-based polymer is 96/4 PDLLA or 50/50 PDLLA, the second PDLLA-based polymer is poly(D,L-lactide-co-caprolactone), and the third PDLLA-based polymer is 50/50 PDLLA; wherein the first PDLLA-based polymer is 50/50 PDLLA or 96/4 PDLLA, the second PDLLA-based polymer is poly(D,L-lactide-co-caprolactone), and the third PDLLA-based polymer is 50/50 PDLLA; wherein the first PDLLA-based polymer is 50/50 PDLLA or 96/4 PDLLA, the second PDLLA-based polymer is 50/50 PDLLA, and the third PDLLA-based polymer is 50/50 PDLLA; wherein the first PDLLA-based polymer is 50/50 PDLLA or 96/4 PDLLA, the second PDLLA-based polymer is poly(D,L-lactide-co-caprolactone), and the third PDLLA-based polymer is poly(D,L-lactide-co-caprolactone); wherein the drug is selected from the group consisting of everolimus, rapamycin, novolimus, zotarolimus, and biolimus; wherein a thickness of the primer layer is 0.2 to 2 microns; and wherein a thickness of the therapeutic layer is 2 to 20 microns; wherein the first PDLLA-based polymer is amorphous.

An embodiment of the present invention includes a method of coating a stent comprising: providing a scaffold including a poly(D,L-lactide)(PDLLA)-based polymer having at least 50% L-enantiomer and at least 4% D-enantiomer, wherein the polymer is amorphous; applying a coating composition to a surface of the scaffold, wherein the coating composition comprises a drug and a coating polymer composed of a PDLLA-based polymer dissolved in a fluid, wherein the fluid is a blend of a good solvent for the coating polymer and a poor solvent for the coating polymer; and removing the solvent from the applied coating composition.

The embodiment may include one or any combination of the following aspects: wherein a ratio of the good solvent to the poor solvent is 90/10 to 10/90 by weight; wherein the good solvent is selected from the group consisting of acetone, methylene chloride, chloroform, 2-butanone, ethyl acetate, methyl acetate, tetrahydrofuran, dioxane, nitropropane, cyclohexanone, butyl benzoate, dimethylformamide, dimethylacetamide, benzyl benzoate, and N-methylpyrrolidone; wherein the poor solvent is selected from the group consisting of pentane, hexane, heptane, cyclopentane, cyclohexane, methanol, ethanol, isopropanol, n-butyl acetate, diisopropyl ketone, and toluene; wherein the poor solvent has a lower boiling point than the good solvent; wherein the poor solvent has a boiling point lower than 55 deg C. and the good solvent has a boiling point greater than 55 deg C.; further comprising repeating the applying and removing steps one or more times; wherein the drug is selected from the group consisting of everolimus, rapamycin, novolimus, zotarolimus, and biolimus; wherein the coating is disposed over at least a portion of the abluminal surface of the scaffold only; wherein a thickness of the coating is between 1 and 5 microns.

An embodiment of the present invention includes a method of coating a stent comprising: providing a scaffold including a scaffold polymer composed of a poly(D,L-lactide)-based polymer having a glass transition temperature (Tg) greater than 37 deg C.; forming a drug coating over at least a portion of the scaffold surface using a coating process, wherein the drug coating comprises a poly(DL-lactide)-based polymer, a drug, and residual solvent from the coating process, and wherein the coated scaffold is at a diameter; and thermally processing the coated scaffold to remove the residual solvent, wherein the thermal processing comprises increasing a temperature of the coated scaffold to a temperature below the Tg of the scaffold polymer followed by reducing the temperature, wherein the thermal processing accelerates physical aging and stabilizes the dimensions of the scaffold, the density of the scaffold polymer, mechanical properties of the scaffold polymer, scaffold properties, or any combination thereof.

The embodiment may include one or any combination of the following aspects: wherein a thickness of the drug coating is greater than 10 microns; wherein the scaffold is at a diameter greater than a targeted deployment diameter during the thermal processing; wherein the scaffold is amorphous; wherein the scaffold polymer is 94/4 PDLLA or 50/50 PDLLA; wherein the coating polymer is 50/50 PDLLA or poly(D,L-lactide-co-caprolactone); wherein the thermal processing reduces residual solvent composition of the coating from greater than 5 wt % to less than 2 wt %; wherein a temperature of the thermal processing is Tg-15 deg C. to Tg; wherein the thermal processing increases the modulus of the scaffold polymer, the radial strength of the scaffold, or both.

An embodiment of the present invention includes a method of coating a stent comprising: providing a scaffold including a scaffold polymer composed of a poly(D,L-lactide)-based polymer having a glass transition temperature (Tg) greater than 37 deg C.; forming a drug coating over at least a portion of the scaffold surface using a coating process, wherein the drug coating comprises a poly(D,L-lactide)-based polymer, a drug, and residual solvent from the coating process; and thermally processing the coated scaffold to remove the residual solvent, wherein the thermal processing comprises increasing a temperature of the coated scaffold to a temperature above the Tg of the scaffold polymer followed by reducing the temperature, wherein the thermal processing reverses physical aging of the scaffold polymer.

The embodiment may include one or any combination of the following aspects: wherein a thickness of the drug coating is greater than 10 microns; wherein the scaffold is at a diameter greater than a targeted deployment diameter; wherein the scaffold is amorphous; wherein the scaffold polymer is 94/4 PDLLA or 50/50 PDLLA; wherein the coating polymer is 50/50 PDLLA or poly(D,L-lactide-co-caprolactone); wherein the thermal processing reduces residual solvent composition of the coating from greater than 5 wt % to less than 2 wt %; wherein a temperature of the thermal processing is Tg-15 deg C. to Tg; wherein the thermal processing decreases the modulus of the scaffold polymer, increase the elongation to break of the scaffold polymer, increases the fracture resistance of the scaffold polymer, or any combination thereof.

An embodiment of the present invention includes a method of treating restenosis in a patient in need thereof, comprising: implanting a bioresorbable stent comprising a scaffold and an antiproliferative drug at a stenotic section of a vessel of a patient; and releasing the antiproliferative drug from the stent, wherein release of the drug is completed or substantially completed prior to the onset of positive remodeling of the section of the vessel.

The embodiment may include one or any combination of the following aspects: wherein the release is 100% completed prior to the onset of positive remodeling; wherein the release of the drug is completed or substantially completed by 2 months or 3 months after deployment of the stent; wherein the release of the drug is completed or substantially completed when a radial strength of the stent is less than 350 mm Hg, or when stent radial strength is 50% of the stent's radial strength directly after deployment; wherein the release of the drug is completed or substantially completed when a number average molecular weight of the scaffold is less than 47 kDa; wherein the release of the drug is completed or substantially completed when scaffold's Mn is 50% of the stent's Mn directly after deployment.

An embodiment of the present invention includes a method of treating restenosis in a patient in need thereof, comprising: implanting a bioresorbable stent comprising a scaffold and an antiproliferative drug at a stenotic section of a vessel of a patient; inhibiting or preventing release of the antiproliferative drug until after early positive remodeling of the section of the vessel is completed; and releasing the drug from the stent after early positive remodeling of the section of the vessel is completed.

The embodiment may include one or any combination of the following aspects: wherein the early positive remodeling is completed when the scaffold is broken up sufficiently to allow freedom of movement of the vessel; wherein the drug is released no earlier than 3 months post-implantation; wherein the drug is released when the number average molecular weight of the scaffold is less than 47 kDa; wherein the drug is released when scaffold's Mn is 50% of the stent's Mn directly after deployment.

An embodiment of the present invention includes a stent comprising: a scaffold comprising a poly(D,L-lactide)(PDLLA)-based polymer having at least 50% L-enantiomer and at least 4% D-enantiomer, wherein the polymer is amorphous; a therapeutic coating disposed over at least a portion of a surface of the scaffold, wherein the therapeutic layer comprises an antiproliferative drug mixed within a coating polymer composed of poly(D,L-lactide) or poly(D,L-lactide-co-caprolactone); and a barrier coating comprising a bioabsorbable polymer over the therapeutic coating to prevent release of the drug from the stent until after early positive remodeling of the section of the vessel is completed.

The embodiment may include one or any combination of the following aspects: wherein the barrier coating is drug-free; wherein the barrier coating is tuned to degrade and allow release of the drug after 3 months; wherein the coating polymer is selected from the group consisting of poly(D,L-lactide), poly(L-lactide), polyglycolide, polycaprolactone, polydioxanone, poly(4-hydroxybutyrate), and copolymers and blends thereof; wherein the coating polymer is selected from the group consisting of aliphatic polyanhydrides, hydrophobic aromatic polyanhydrides, polyester amides, poly(ortho esters), and polyketals.

### BRIEF DESCRIPTION OF THE DRAWINGS

FIG. 1 depicts an exemplary scaffold.

FIG. 2 depicts a cross-section of a stent surface with a polymer and drug layer.

FIG. 3 depicts a cross-section of a strut of a scaffold with a primer layer over the strut and a therapeutic layer over the primer layer.

### DETAILED DESCRIPTION OF THE INVENTION

The present invention relates to drug-eluting coatings on poly(DL-lactide)-based stents or scaffolds. In particular, a stent body may be a scaffold composed of a bioresorbable polymer and the therapeutic coating includes a bioresorbable polymer carrier and a drug. In general, a radially expandable stent or scaffold can have virtually any structural pattern that is compatible with a bodily lumen in which it is implanted. In certain aspects, a stent is composed of a pattern or network of circumferential rings and longitudinally extending interconnecting structural elements of struts or bar arms. The struts are arranged in patterns, which are designed to contact the lumen walls of a vessel and to maintain vascular patency.

An exemplary structure of a stent body or scaffold is shown in FIG. 1. FIG. 1 depicts a stent 10 which is made up of struts 12. Stent 10 has interconnected cylindrical rings 14 composed of undulating struts. Cylindrical rings 14 are connected by linking struts or links 16. The embodiments disclosed herein are not limited to fabricating stents or to the stent pattern illustrated in FIG. 1. The embodiments are easily applicable to other stent patterns and other devices. The variations in the structure of patterns are virtually unlimited. The outer diameter of a fabricated stent (prior to crimping and deployment) may be between 0.2-5.0 mm. For coronary applications, a fabricated stent diameter is 2.5-5 mm. The length of the stents may be between about 6-38 mm or more depending on the application.

A polymer coating on the surface of a stent body or scaffold may also include a biodegradable polymer. The biodegradable polymer may be a carrier for an active agent or drug. The coating polymer may be bioresorbable.

A radial thickness or thickness of the stent body or scaffold may be 80 to 100 microns, 90 to 110 microns, 100 to 120 microns, 120 to 140 microns, 140 to 160 microns, or greater than 160 microns.

The coating is typically much thinner than the struts of the scaffolding, for example, the coating can be 1 to 20 microns, 1 to 10 microns, 10 to 15 microns, 1 to 3 microns, 3 to 10 microns, or 10 to 20 microns. In general, it is desirable for the radial thickness to be as low as possible to avoid interference with blood flow.

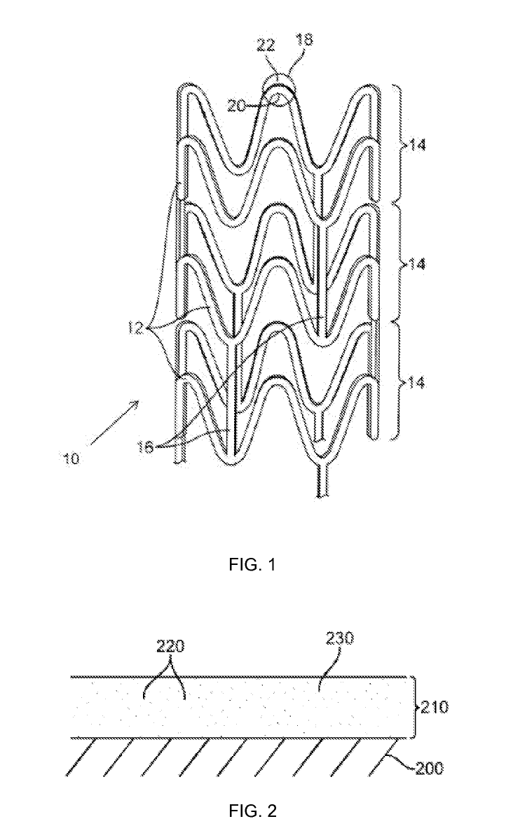

FIG. 2 depicts a cross-section of a stent surface with a polymer and drug coating layer 210 over a substrate 200. Coating layer 210 includes a drug 220 dispersed in a coating polymer 230. A substrate or scaffold can be metallic, polymeric, ceramic, or other suitable material.

A biodegradable stent may be fabricated from a tube with a thin wall initially having no holes or voids. The pattern of structural elements may be formed by laser machining. Material is removed from selected regions of the tube which results in the pattern of structural elements.

The manufacturing process for a bioresorbable stent may include several steps. A polymeric tube may be formed using melt processing such as extrusion or injection molding. Prior to laser machining, the tube may be processed to modify its mechanical properties that also improve stent properties such as radial strength and resistance to fracture. Such processes may include radially deforming the tube. The scaffold pattern may then be formed by laser machining. A therapeutic coating may be formed over the scaffold.

A polymer coating over a scaffold may be formed using various solution techniques which involve application of a coating composition including a polymer, drug, and solvent to the scaffold surface, followed by removing the solvent. The coating composition can be applied to a scaffold substrate by various methods, such as, dip coating, brushing, or spraying. The aspects of the present invention are not limited to any particular application or deposition technique. In particular, spray coating a stent typically involves mounting or disposing a stent on a support, followed by spraying a coating composition from a nozzle onto the mounted stent. Solvent is removed from the deposited coating composition to form the coating. There typically is some residual solvent remaining in the coating after the solvent removal or solvent removal steps. As discussed in more detail below, solvent removal can be performed through evaporation at room or ambient temperature or by heating or exposing a coated stent to a temperature above room temperature. Room or ambient temperature may be between 20 and 30 deg C. and any temperature or range in between.

If a coating layer of a target thickness (or mass) is formed with a single application step and then followed by solvent removal, the coating layer that results can be nonuniform, include coating defects, or both. Stents, particularly those for coronary use, comprise an intricate stent pattern with small dimensions. If too much coating composition is applied all at once, it could form webs, pools, or strands in the stent pattern. Instead of a desired conformal coating, a highly non-uniform coating results. Therefore, a coating of a target thickness (or mass) is preferably formed with two or more cycles or passes of a coating composition application, such as spraying. After each cycle or pass, a solvent removal or drying step is performed. The solvent removal step after each pass is referred to as interpass drying. A cycle or pass refers to the application of a coating composition without an intervening solvent removal step, such as blowing warm air on the stent. In spraying, a cycle or pass can include directing the spray plume over the length of a stent one or more times. After each coating composition application pass, the application of coating composition on the substrate is stopped, which is followed by interpass solvent removal. An exemplary coating process is described in US 2010/0323093.

The above processes are typically performed with the scaffold at a diameter larger than that required for delivery into vessel. After coating, the coated scaffold may be reduced in diameter or crimped to a diameter suitable for delivery over a support such a delivery balloon. The crimped scaffold may then be subjected to a sterilization process such as e-beam or gamma radiation. The stent is implanted in a patient by positioning the crimped scaffold at a site of stenosis in a blood vessel and expanding the stent with the delivery balloon.

A radially expandable scaffold or stent body should have the ability to hold open narrowed portions of blood vessels. Therefore, the scaffold should possess a radial strength in an expanded state that is sufficiently high and sustainable to maintain the expanded vessel size for a period of weeks or months. A polymer or polymer formulation for a scaffold should be stiff and strong after processing into a scaffold under physiological conditions within a human body. Polymer or polymer formulations that have a glass transition temperature (Tg) in a dry state sufficiently above human body temperature (approximately 37 deg C.), particularly those that include semicrystalline polymers, meet the above criterial. Poly(D,L-lactide)-based polymers are examples of such polymers.

The polymer or polymer formulation of a scaffold of the present invention may include poly(D,L-lactide)-based polymer which include a poly(D,L-lactide) (PDLLA) component having a constitutional unit weight percentage L-lactide and D-lactide units of 50/50 to 96/4, such as 50/50 or 96/4 poly(D,L-lactide). The term “unit” or “constitutional unit” refers to the composition of a monomer as it appears in a polymer. The poly(D,L-lactide)-based polymer may be a PDLLA homopolymer or a copolymer of PDLLA such as poly(D,L-lactide-co-caprolactone), poly(D,L-lactide-co-glycolide), poly(D,L-lactide-co-L-lactide), poly(lactic-co-glycolic-co-gluconic acid). The copolymers may be random or block copolymers. The polymer formulation may further include a blend of a PDLLA-based polymer and another polymer such as homopolymers and copolymers including polydioxanone, polyethylene oxide, polyethylene glycol, poly(butylene succinate), poly(trimethylene carbonate), poly(butylene succinate), or any combination thereof.

The PLLA-co-CL copolymer can have weight or mole percentage of caprolactone units of 1 to 25%, or more narrowly, to 5%, 5 to 10%, 1 to 3%, 3 to 5%, 5 to 8%, 8 to 10%, 10 to 15%, or 15 to 25%. PLGA copolymer can have molar or weight percentages of L-lactide or D,L-lactide and glycolide units, of 1 to 90%, or more narrowly, 1 to 10%, 10 to 25%, 25 to 50%, 50 to 75%, or 75 to 90%. Exemplary PLGA polymer compositions (% D,L-lactide:% glycolide) are 90:10, 75:25, 50:50, 25:75, and 10:90.

Embodiments of the invention include a scaffold made substantially or completely of the PDLLA-based polymer. “Substantially” may correspondent to greater than 90 wt %, greater than 95 wt %, or greater than 99 wt %. The scaffold may have a composition of 90 to 95% or 95 to 99% of the polymer formulation.

The scaffold or the polymer formulation of the scaffold may be amorphous or substantially amorphous. “Amorphous” or “substantially amorphous” means less than 5%, less than 1%, less than 2%, less than 4%, or 1 to 5% crystallinity.

The polymer for a polymer carrier of a therapeutic coating over the scaffold may include a PDLLA-based polymer as described above. Exemplary combinations of scaffold polymer and coating polymer include 50/50 PDLLA and 50/50 PDLLA; 96/4 PDLLA and 50/50 PDLLA; and 50/50 PDLLA and 96/4 PDLLA.

A drug may be mixed or dispersed throughout the polymer carrier. The drug may be 20 to 80 wt % of the therapeutic layer, or more narrowly, 30 to 70 wt %, 40 to 60 wt %, 45 to 55 wt %, or 50% of the therapeutic layer. Exemplary drugs include rapamycin, everolimus, novolimus, zotarolimus, deforolimus, temsirolimus, merilimus, umirolimus or biolimus.

Certain embodiment of the present invention include a stent including a scaffold including a poly(D,L-lactide)(PDLLA)-based polymer composed of at least 50% L-enantiomer and at least 4% D-enantiomer. The polymer may be amorphous. The stent further includes a therapeutic coating or layer disposed over at least a portion of a surface of the scaffold. The therapeutic layer includes a drug mixed within a coating polymer composed of a poly(D,L-lactide) or poly(D,L-lactide-co-caprolactone). The PDLLA-based polymer may include 50/50 PDLLA, 96/4 PDLLA, or a copolymer thereof. A copolymer may include poly(96/4 D,L-lactide-co-caprolactone) or poly(50/50 D,L-lactide-co-caprolactone).

The coating is disposed only over selected portions of the scaffold surface. In one embodiment, the luminal surface is free of coating and the coating is over the entire abluminal surface of the scaffold and part of the sidewall surfaces. In another embodiment, the coating is over at least a portion of the abluminal surface of the scaffold only with the sidewalls and luminal surface free of coating.

The scaffold may further include unpolymerized L- or D,L-lactide monomer dispersed within the scaffold. The lactide content of the scaffold may be 0.001 to 1 wt %, or more narrowly, 0.01 to 1 wt %, 0.1 to 0.5 wt %, 0.5 to 0.7 wt %, or 0.7 to 1 wt % of the scaffold. The presence of lactide monomer accelerates the degradation of the scaffold and shortens the time required for dismantling or break-up of the scaffold.

Further embodiments of the present invention are directed at enhancing adhesion of a PDLLA-based therapeutic coating on a PDLLA-based scaffold. The invention includes an intermediate layer or primer layer between the scaffold and a therapeutic layer. As the therapeutic layer incorporates immiscible drug as well as polymer, the primer layer provides properties intermediate between the scaffold polymer and drug layer with a high weight percent of drug. The drug component of the PDLLA-based therapeutic coating can also occupy the interface between the coating and PDLLA-based scaffold. This can reduce the coating adhesion.

Embodiments of a stent are a scaffold including a polymer including a first poly(D,L-lactide)(PDLLA)-based polymer of the scaffold and primer layer including a second PDLLA-based polymer disposed on a surface of the scaffold. In one embodiment, the primer layer covers an entire surface of the scaffold including sidewalls, abluminal, and luminal surfaces. The primer layer is free of a drug or therapeutic agent. A therapeutic layer including a third PDLLA-based polymer is disposed over at least a portion of the primer layer. The therapeutic layer includes a drug mixed or dispersed in the third PDLLA-based polymer. In some embodiments, the scaffold is amorphous.

In some embodiments, the therapeutic layer is disposed only over selected portions of the primer surface. In one embodiment, the luminal surface is free of the therapeutic layer and the therapeutic layer is over the entire abluminal surface of the scaffold and part of the sidewall surfaces with the primer layer between the therapeutic layer and scaffold surface. In another embodiment, the therapeutic layer is over at least a portion of the abluminal surface of the scaffold only with the primer layer between the therapeutic layer and the scaffold surface and the sidewalls and luminal surfaces are free of the therapeutic layer.

The first or scaffold PDLLA-based polymer may be 50/50 PDLLA or 96/4 PDLLA. The second or primer PDLLA-based polymer may be 50/50 PDLLA or poly(D,L-lactide-co-caprolactone). The third or therapeutic PDLLA-based polymer may be 50/50 PDLLA.

In one embodiment, the scaffold polymer is 50/50 PDLLA, the primer polymer is poly(D,L-lactide-co-caprolactone), and the therapeutic polymer is 50/50 PDLLA.

In another embodiment, the scaffold polymer is 96/4 PDLLA, the primer polymer is poly(D,L-lactide-co-caprolactone), and the therapeutic polymer is 50/50 PDLLA.

In another embodiment, the scaffold polymer is 96/4 PDLLA, the primer polymer is 50/50 PDLLA, and the therapeutic polymer is 50/50 PDLLA. \

In another embodiment, the scaffold polymer is 96/4 PDLLA, the primer polymer is poly(D,L-lactide-co-caprolactone), and the therapeutic polymer is poly(D,L-lactide-co-caprolactone).

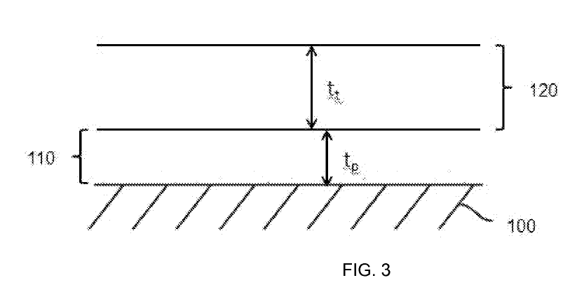

FIG. 3 depicts a cross-section of a strut 100 of a scaffold with a primer layer 110 over strut 100 and a therapeutic layer 120 over primer layer 110. Primer layer 110 has a thickness tₚ and the therapeutic layer 120 has thickness tₜ. A thickness of the primer layer may be 0.2 to 5 microns and a thickness of the therapeutic layer may be 1 to 15 microns.

Forming a coating composed of a PDLLA-based polymer on a PDLLA-based scaffold can be challenging when using a solvent-based application process. For example, acetone may be useful as a solvent for forming a coating of 50/50 PDLLA or poly(D,L-lactide-co caprolactone). However, a suitable solvent for the 50/50 PDLLA coating polymer may also be a good solvent for the PDLLA-based scaffold. Therefore, a coating process will expose the PDLLA-based scaffold to a good or strong solvent which could damage or degrade the scaffold due to dissolution, distortion or modification of the scaffold polymer morphology.

The potential for damage is especially high for relatively thick coatings (e.g., 10 microns or more) which result in exposure of the scaffold to solvent for an extended period of time. The exposure time also depends on the type of application process. A direct fluid application does not remove solvents as fast as a spray process which involves multiple passes with inter-pass drying that removes solvent between passes.

The present invention includes solvent-based application methods that use a solvent blend of a good solvent for a PDLLA-based polymer combined with a poor solvent or non-solvent for the PDLLA-based coating polymer. The poor solvent may also be more volatile than the good solvent. The blend reduces the harmful solvent exposure to the scaffold.

Whether a solvent is a good solvent or a poor solvent for PDLLA-based polymers may be estimated by the Hildebrand Solubility Parameter, or cohesive energy density 6, combined with the degree of Hydrogen bonding of the solvent (poor, moderate, strong). A poor solvent for PDLLA-based polymers may be characterized by a solubility parameter that is <9.1 (cal/cm³)^{1/2} or >12.1 (cal/cm³)^{1/2} or which has a strong hydrogen bonding force. Conversely, a good solvent for PDLLA-based polymers may be characterized by a solubility parameter which lies in the range of 9.1≦δ≦12.1 (cal/cm³)^{1/2} and which the hydrogen bonding force is classified as poor to moderate. Embodiments of a coating method may include providing a scaffold including a PDLLA-based polymer composed of at least 50% L-enantiomer and at least 4% D-enantiomer. A coating composition is applied to a surface of the scaffold such that the coating composition includes a drug and a coating polymer composed of a PDLLA-based polymer dissolved in a solvent blend. The solvent blend is a blend of a good solvent for the coating polymer and a poor solvent for the coating polymer. The method further includes removing the solvent blend from the applied coating composition. The scaffold polymer may be 96/4 PDLLA or 50/50 PDLLA and the coating polymer may be poly(D,L-lactide-co-caprolactone) or 50/50 PDLLA.

A solvent blend may have a ratio by weight of the good solvent to the poor solvent of 90/10 (90 wt % good solvent, 10 wt % poor solvent) to 10/90, or more narrowly, 90/10 to 70/30, 70/30 to 30/70, 70/30 to 50/50, 50/50 to 70/30, or 30/70 to 10/90. The ratio can be adjusted according to the degree of solvent exposure in a particular coating method, the volatility of the solvents, the strength of the good solvent, or any combination. In a coating application method such as spray coating in which the solvent exposure from a spray pass may be less than 5 to 30 seconds, a higher ratio of good to poor solvent may be used, such as greater than 50/50 or greater than 70/30. For a direct coating method with higher exposure times of 1 to 5 min, a lower ratio of good to poor solvent may be used, such as less than 50/50 or less than 30/70.

Poor solvents would be selected to have a lower boiling point or a higher vapor pressure at ambient temperature than a good solvent. In some embodiments, the selected poor solvent has a boiling point lower than 55 deg C. and the good solvent has a boiling point greater than 55 deg C.

Good solvents for PDLLA-based polymers include acetone, methylene chloride, chloroform, 2-butanone, ethyl acetate, methyl acetate, dioxane, dimethylformamide, cyclohexanone, N-methylpyrrolidone, butyl benzoate, nitropropane, dimethylacetamide, benzyl benzoate, and tetrahydrofuran. Poor solvents for PDLLA-based polymers include pentane, hexane, heptane, cyclohexane, cyclopentane, methanol, ethanol, isopropanol, n-butyl acetate, diisopropyl ketone, and toluene.

Exemplary solvent blends include pentane/acetone or cyclohexane/MEK.

Several direct application methods can be used to achieve an abluminal coating. In one technique, the stent pattern is followed by an ink-jet applicator that applies coating in droplet form to the abluminal surfaces. In another embodiment, a direct fluid dispense is used where an applicator tip traces the stent pattern, applying coating solution to the abluminal surfaces. In yet another embodiment, a roll coating technique is used to apply solution to the ablumenal surfaces. To accomplish this, a uniform coating of the coating solution is formed on a cylindrical applicator and this cylindrical applicator is brought into contact with the abluminal surface of the stent with a rolling action. With any of these techniques, the coating applied can be less than the final targeted value. This exposes the underlying scaffold to less solvent. After each pass, the coating can have solvent removed by drying at ambient temperature, applying forced air or inert gas, or by baking in an oven. Following the drying step, another abluminal coating layer can be applied. Multiple abluminal layers may be applied by these direct application methods.

After formation of a polymer and drug layer on a PDLLA-based scaffold, a thermal treatment step may be used to remove residual solvent from the coating. Baking the coated scaffold in an oven may be the thermal treatment. The thermal treatment is especially important for thick coatings (e.g., greater than 10 microns). This baking step may affect the scaffold.

The thermal treatment step may also be used to condition or modify the scaffold to improve the scaffold properties for improved performance once implanted.

The PDLLA-based scaffold may be amorphous or at least 50 to 60% amorphous and have a glass transition temperature (Tg) above ambient temperature and body temperature. Because of its high amorphous content, the PDLLA-based scaffold is susceptible to physical aging both prior to the coating step and after the coating step all the way until implantation. Physical ageing is a process that occurs in the amorphous phase of a polymer when it is stored below its Tg. In this process, the amorphous phase undergoes densification to more of an equilibrium state. For a polymer scaffold, physical aging can also occur and translates into changes in physical and thermodynamic properties of the polymer of the scaffold with time. Physical ageing is of particular relevance for amorphous and semi-crystalline polymers that include amorphous regions that have glass transition temperatures (T_{g}) above their normal storage temperature, which is typically ambient or room temperature, i.e., from about 15° C. to about 35° C., or more narrowly, 20° C. to about 30° C., 25° C., or about 30° C. At temperatures below Tg, semi-crystalline and amorphous polymers are not in thermodynamic equilibrium and physical properties, such as specific volume, enthalpy and entropy which are greater than the equilibrium values decrease towards the equilibrium values at rates which decrease with the degree of undercooling below the Tg. Physical ageing can make the scaffold brittle or stiffer and more susceptible to fracture when the scaffold is plastically deformed during crimping. The changes in physical properties that occur during physical aging include an increase in density, increase in modulus, decrease in compliance, increase in stiffness, and a decrease in ultimate strength.

Physical aging of a PDLLA-based scaffold during storage can be undesirable even if it does not harm performance since time-dependent change in properties result in inconsistent product quality. Thus, in one embodiment, the thermal treatment may accelerate physical aging to reduce or eliminate time dependence in properties after the thermal treatment. As a result the product quality will be consistent.

In such embodiments, a method may include forming a drug coating over at least a portion of the PDLLA-based scaffold surface using a coating process. The drug coating includes a poly(D,L-lactide)-based polymer, a drug, and residual solvent from the coating process.

The coated scaffold is thermally processed to remove the residual solvent and to stabilize the scaffold. The thermal processing includes increasing a temperature of the coated scaffold to a temperature below a glass transition temperature (Tg) of the scaffold polymer followed by reducing the temperature. The thermal processing stabilizes properties of the scaffold during storage through acceleration of physical aging of the polymer. In particular, the thermal processing stabilizes the dimensions of the scaffold, the density of the scaffold polymer, mechanical properties of the scaffold polymer, scaffold properties, or any combination thereof.

The Tg of a PDLLA-based polymer or PDLLA-based scaffold polymer may be greater than 45 deg C., 45 to 60 deg C., 45 to 48 deg C., 48 to 58 deg C., or 55 to 60 deg C. However, the actual value of the scaffold Tg may depend on its processing history.

The coated scaffold is preferably at a diameter greater than its intended or targeted deployment diameter in a blood vessel during the thermal processing. The thermal processing diameter may correspondent to the diameter at which the scaffold was fabricated, e.g., the diameter during the laser machining process. Stabilizing the scaffold at a diameter greater than the targeted deployment diameter is expected to reduce or prevent recoil upon deployment and increase the scaffold post-dilatation capability. In another embodiment, the scaffold is fabricated and coated at a smaller diameter than its intended maximum post-dilatation size. The scaffold is then expanded and heated at a temperature above the Tg to set the amorphous phase to the new larger diameter, and remove any amorphous phase memory of the as-cut diameter. Optionally, the scaffold is heated while being expanded to the new larger diameter.

In some embodiments, the thermal processing reduces residual solvent composition of the coating from greater than 5 wt % to less than 3 wt %, less than 2 wt %, 1 to 3 wt %, 1 to 2 wt %, or less than 1 wt %.

In an embodiment, the thickness of the drug coating is greater than 10 microns. The temperature of the thermal processing for acceleration of physical aging may be Tg-10 deg C. to Tg, Tg-15 deg C. to Tg-5 deg C., or Tg-15 deg C. to Tg. The time of the thermal processing will depend on the degree of residual solvent desired and the degree of stabilization or change in scaffold property(ies) desired, or both. Exemplary thermal processing times are 1 to 5 min, 5 to 10 min, 10 to 30 min, 30 min to 1 hr, or greater than 1 hr.*

The dimensions of the scaffold refer to the diameter and shape of the scaffold. The mechanical properties refer, for example, to the modulus, strength, and elongation at break. The scaffold properties refer, for example, to the radial strength of the scaffold and a maximum post-dilatation diameter. Stabilize may correspond to less than 1%, less than 5%, or less than 10% change over a period of 1 week, 1 month, 3 months, 6 months, or a year at a normal storage temperature, e.g., 25 deg C.

The thermal processing step may cause increase in the modulus of the scaffold polymer, the radial strength of the scaffold, or both. The modulus may increase by at least 1%, 1 to 2%, 2 to 5%, or greater than 5%.

In another embodiment, the thermal processing step can reverse physical aging in the PDLLA-based scaffold polymer that occurs during its processing history. Thermally processing at a temperature greater than a Tg of the scaffold polymer can reverse physical aging. In this embodiment, the coated scaffold diameter is also preferably greater than its intended or targeted deployment diameter in a blood vessel during the thermal processing. Reversing physical aging at a diameter greater than a targeted deployment diameter may reduce or prevent recoil upon deployment.

The thermal processing may include increasing a temperature of the coated scaffold to a temperature above the glass transition temperature (Tg) of the scaffold polymer followed by reducing the temperature. The temperature of the thermal processing for reversing physical aging may be Tg to Tg+10 deg C., Tg+5 deg C. to Tg+15 deg C., or Tg to Tg+15 deg C. The time of the thermal processing will depend on the degree of residual solvent removal desired and the degree of reversal of physical aging or change in scaffold property(ies) desired, or both. Exemplary thermal processing times are 1 to 5 min, 5 to 10 min, 10 to 30 min, 30 min to 1 hr, or greater than 1 hr.

Thermally processing to reverse physical aging of the scaffold polymer can decrease the modulus of the polymer, increase the elongation to break of the polymer, increase the fracture resistance of the polymer, or any combination thereof. The modulus may decrease by at least 1%, 1 to 5%, 5 to 10%, or greater than 10%. The increase in fracture resistance may result in a reduction in fracture when the scaffold his crimped. As a result, the radial strength when the scaffold is deployed may be higher. The thermal processing may cause no change in crystallinity in the scaffold polymer.

Further embodiments relate to facilitating the positive vascular remodeling process with a bioabsorbable scaffold. Vascular remodeling refers generally to a persistent change in vessel size which has been identified as the primary determinant of lumen size in the presence of stable lesions. Circulation, 2000; 102: 1186-1191. The term arterial remodeling may refer to a change in vessel size (or cross-sectional area) within the external elastic lamina. Negative or inward remodeling denotes a reduction in vessel size while positive or outward remodeling denotes an increase in vessel size.

A bioabsorbable scaffold implanted at a stenotic region of a blood vessel provides temporary mechanical support or patency after deployment to prevent inward remodeling. During this period of support, a healing process occurs which is believed to stabilize the vessel walls. With bioresorption, the scaffold gradually loses strength and stiffness, develops structural discontinuities, and degrades within the vessel. This gradual loss of supports allows positive remodeling to naturally occur. During gradual loss of support, the constraint of the scaffold on movement of the vessel is gradually eliminated. A period of radial support of three months is believed to be necessary and sufficient to prevent negative inward remodeling after deployment and result in positive remodeling that allows the vessel to support itself.

The onset of natural positive remodeling may coincide with the reduction in strength and stiffness of the scaffold and the development of discontinuities within the scaffold structure. Therefore, after about 3 to 4 months post-deployment, the onset of positive remodeling may be related to the radial strength of the scaffold. Since the reduction in radial strength and development of discontinuities within the scaffold is due to reduction in the scaffold polymer molecular weight, the molecular weight of the scaffold, the onset of positive remodeling can be related to the molecular weight of the scaffold. For example, the onset of positive remodeling may correlate to a radial strength of 350 to 1000 mm Hg, or a reduction in radial strength to a level that constitutes 50% to 15% of the scaffold's radial strength directly after deployment. Alternatively, the onset of positive remodeling may correlate to a number average molecular weight (Mn) of the scaffold polymer of 47 to 20 kDa, or a reduction in Mn to a level that constitutes 50% to 25% of the scaffold's Mn directly after deployment

If molecular weight remains within 10% over the time course of positive remodeling, the onset of positive remodeling can instead be related to the number of discontinuities (or fractures) developing over time within the scaffold structure. For example, the onset of positive remodeling may correlate to state wherein 35% to 60% of the scaffold crests exhibit fractures.

Trauma to the vessel from angioplasty and/or scaffold deployment causes inward remodeling or restenosis due to neointimal growth. Thus, drug elution of an antiproliferative drug from the scaffold may be used to control this neointimal growth. However, release of drugs to prevent restenosis may interfere with the natural positive remodeling since drug may cause cellular disruption, particular re-endothelialization which is an important factor at the early stages of positive remodeling. The early stages remodeling may correspond to the gradual transition from vessel support to complete freedom of movement of the vessel. Aspects of the invention include two approaches for addressing this problem.

In a first approach, all or most of the drug release from the stent occurs prior to and completes before the remodeling process begins or before the onset of positive remodeling. In a second approach, drug elution can be timed to only elute after early inward remodeling has completed or substantial remodeling has initiated. The drug elution after early remodeling has completed may manage further neo-intima growth, but still allow for unhindered remodeling.

In the first approach, a method of treating stenosis in a patient in need thereof includes releasing an antiproliferative drug from the stent, such that the drug is no longer being released when the positive remodeling starts. Therefore, any cellular disruption due to the drug is avoided during the positive remodeling. In such embodiments, the drug release may be tuned to be Substantially completed or fully completed prior to the onset of positive remodeling. “Substantially completed” may refer to 80 to 99%, or more narrowly, 80 to 90%, 90 to 95%, 95 to 99%, or 99% to below 100% of complete drug elution. Complete elution may refer to the total drug dose or weight of drug on the stent or complete elution from drug release profile (i.e., cumulative drug elution vs. time) from an in vitro drug elution test.

In such embodiments, the drug release may be tuned to be mostly or fully complete prior to 2 months, 3 months, or 2 to 3 months post-deployment of the stent. Alternatively, the drug release may be tuned to be mostly or fully complete prior to the onset of positive remodeling via a corresponding decrease in the radial strength, molecular weight, or both.

In the second approach, a method of treating restenosis in a patient in need thereof includes delaying or partially delaying the release of an antiproliferative drug on the stent implanted in a stenotic section of a blood vessel. The release may be delayed for a period of time after implantation of the stent until at least after early remodeling of the section is completed. The drug may be released from the stent after the period of time.

The completion of early remodeling may correspond to a time when the scaffold is weakened or sufficiently discontinuous to allow freedom of movement of the vessel. Structurally this may correspond to broken struts throughout the scaffold to the point that the scaffold cannot restrain radial movement of the vessel. This time may correspond to a time 3 months post-deployment. The time may also correspond to a time when the molecular weight of the scaffold polymer is below 47 kDa, or when molecular weight has reduced to 50% of the scaffold's Mn directly after deployment.

Additionally, the time of completion of early remodeling may be assessed from pre-clinical or clinical studies. Early remodeling may be detected from changes in vessel volume, area, and diameter which can be measured using conventional analytical techniques such as OCT and IVUS.

It has been observed by the inventors that the Tg of the scaffold polymer changes with time after implantation. The Tg initially increases during a time period, then remains constant for a period of time, and then declines. It is believed that the decline represents a change in mobility of the chains leading to decreased radial strength. In some embodiments, the release of the drug is completed or substantially completed when the Tg decline due to degradation of the polymer begins to drop below the Tg right before implantation. The Tg right before implantation may correspond to a peak Tg following physical aging of the scaffold such as during storage. Drug release from a therapeutic coating on a stent may be partially or fully delayed using a bioresorbable topcoat over the therapeutic layer until early remodeling has substantially occurred. The biodegradable topcoat may be drug-free and tuned to degrade and allow drug elution to the vessel after the early remodeling is complete, for example, 3 months post-implantation, when the scaffold is discontinuous and no longer provides mechanical support to the vessel.

The topcoat may be disposed over all or part of the therapeutic layer. Various parameters of the topcoat layer may be tuned or adjusted to obtain the desired delay of the drug release. The parameters include the thickness of the coating, the degradation rate of the topcoat polymer, and diffusion rate of the drug through the topcoat polymer. A thickness of the topcoat layer may be 1 to 10 or 1 to 5 microns.

The topcoat polymer may be bulk-eroding polymer such as poly(D,L-lactide), poly(L-lactide), polyglycolide, polycaprolactone, polydioxanone, poly(4-hydroxybutyrate), and copolymers and blends thereof. The topcoat polymer may include surface eroding polymers such as aliphatic polyanhydrides, hydrophobic aromatic polyanhydrides, polyester amides, poly(ortho esters), and polyketals. Exemplary polyanhydrides include poly(sebacic acid-hexadecanioic acid anhydride) and poly(sebacic acid-1,3-bis(p-carboxyphenoxy)propane anhydride).

The drug in the aspects of the present invention includes an antiproliferative, anti-inflammatory or immune modulating, anti-migratory, anti-thrombotic or other pro-healing agent or a combination thereof. The anti-proliferative agent can be a natural proteineous agent such as a cytotoxin or a synthetic molecule or other substances such as actinomycin D, or derivatives and analogs thereof (manufactured by Sigma-Aldrich 1001 West Saint Paul Avenue, Milwaukee, Wis. 53233; or COSMEGEN available from Merck) (synonyms of actinomycin D include dactinomycin, actinomycin IV, actinomycin I1, actinomycin X1, and actinomycin C1), all taxoids such as taxols, docetaxel, and paclitaxel, paclitaxel derivatives, all olimus drugs such as macrolide antibiotics, rapamycin, everolimus, novolimus, myolimus, deforolimus, umirolimus, biolimus, merilimus, temsirolimus structural derivatives and functional analogues of rapamycin, structural derivatives and functional analogues of everolimus, FKBP-12 mediated mTOR inhibitors, perfenidone, prodrugs thereof, co-drugs thereof, and combinations thereof. Representative rapamycin derivatives include 40-O-(3-hydroxy)propyl-rapamycin, 40-O-[2-(2-hydroxy)ethoxy]ethyl-rapamycin, or 40-O-tetrazole-rapamycin, 40-epi-(N1-tetrazolyl)-rapamycin (ABT-578 manufactured by Abbott Laboratories, Abbott Park, Ill.), prodrugs thereof, co-drugs thereof, and combinations thereof.

The anti-inflammatory agent can be a steroidal anti-inflammatory agent, a nonsteroidal anti-inflammatory agent, or a combination thereof. In some embodiments, anti-inflammatory drugs include, but are not limited to, novolimus, myolimus, alclofenac, alclometasone dipropionate, algestone acetonide, alpha amylase, amcinafal, amcinafide, amfenac sodium, amiprilose hydrochloride, anakinra, anirolac, anitrazafen, apazone, balsalazide disodium, bendazac, benoxaprofen, benzydamine hydrochloride, bromelains, broperamole, budesonide, carprofen, cicloprofen, cintazone, cliprofen, clobetasol propionate, clobetasone butyrate, clopirac, cloticasone propionate, cormethasone acetate, cortodoxone, deflazacort, desonide, desoximetasone, dexamethasone dipropionate, diclofenac potassium, diclofenac sodium, diflorasone diacetate, diflumidone sodium, diflunisal, difluprednate, diftalone, dimethyl sulfoxide, drocinonide, endrysone, enlimomab, enolicam sodium, epirizole, etodolac, etofenamate, felbinac, fenamole, fenbufen, fenclofenac, fenclorac, fendosal, fenpipalone, fentiazac, flazalone, fluazacort, flufenamic acid, flumizole, flunisolide acetate, flunixin, flunixin meglumine, fluocortin butyl, fluorometholone acetate, fluquazone, flurbiprofen, fluretofen, fluticasone propionate, furaprofen, furobufen, halcinonide, halobetasol propionate, halopredone acetate, ibufenac, ibuprofen, ibuprofen aluminum, ibuprofen piconol, ilonidap, indomethacin, indomethacin sodium, indoprofen, indoxole, intrazole, isoflupredone acetate, isoxepac, isoxicam, ketoprofen, lofemizole hydrochloride, lomoxicam, loteprednol etabonate, meclofenamate sodium, meclofenamic acid, meclorisone dibutyrate, mefenamic acid, mesalamine, meseclazone, methylprednisolone suleptanate, momiflumate, nabumetone, naproxen, naproxen sodium, naproxol, nimazone, olsalazine sodium, orgotein, orpanoxin, oxaprozin, oxyphenbutazone, paranyline hydrochloride, pentosan polysulfate sodium, phenbutazone sodium glycerate, pirfenidone, piroxicam, piroxicam cinnamate, piroxicam olamine, pirprofen, prednazate, prifelone, prodolic acid, proquazone, proxazole, proxazole citrate, rimexolone, romazarit, salcolex, salnacedin, salsalate, sanguinarium chloride, seclazone, sermetacin, sudoxicam, sulindac, suprofen, talmetacin, talniflumate, talosalate, tebufelone, tenidap, tenidap sodium, tenoxicam, tesicam, tesimide, tetrydamine, tiopinac, tixocortol pivalate, tolmetin, tolmetin sodium, triclonide, triflumidate, zidometacin, zomepirac sodium, aspirin (acetylsalicylic acid), salicylic acid, corticosteroids, glucocorticoids, tacrolimus, pimecorlimus, prodrugs thereof, co-drugs thereof, and combinations thereof.

These agents can also have anti-proliferative and/or anti-inflammatory properties or can have other properties such as antineoplastic, antiplatelet, anti-coagulant, anti-fibrin, antithrombonic, antimitotic, antibiotic, antiallergic, antioxidant as well as cystostatic agents. Other active agents which are currently available or that may be developed in the future are equally applicable.

The “glass transition temperature,” Tg, is the temperature at which the amorphous domains of a polymer change from a brittle vitreous, glassy state to a solid deformable, rubbery or ductile state at atmospheric pressure. In other words, the Tg corresponds to the temperature where the onset of segmental motion in the chains of the polymer occurs. When an amorphous or semi-crystalline polymer is exposed to an increasing temperature, the coefficient of expansion and the heat capacity of the polymer both increase as the temperature is raised, indicating increased molecular motion. As the temperature is increased, the heat capacity increases. The increasing heat capacity corresponds to an increase in heat dissipation through movement. Tg of a given polymer can be dependent on the heating rate and can be influenced by the thermal history of the polymer as well as its degree of crystallinity. Furthermore, the chemical structure of the polymer heavily influences the glass transition by affecting mobility. The Tg can be determined as the approximate midpoint of a temperature range over which the glass transition takes place. [ASTM D883-90]. The most frequently used definition of Tg uses the energy release on heating in differential scanning calorimetry (DSC). As used herein, the Tg refers to a glass transition temperature as measured by differential scanning calorimetry (DSC) at a 20° C./min heating rate. Unless stated otherwise, values for “Tg” refer to an upper limit for Tg (E.g., for poly(L-lactide) and the Tg when the material is dry. Poly(L-lactide) has a glass transition temperature range of between about 55 to 60 Deg. C. “Tg” for poly(L-lactide), for purposes of this disclosure, Tg is 60 Deg. C.), or up to 65 Deg. C. for a strain hardened tube. The glass transition temperature is a function of chain flexibility. The glass transition occurs when there is enough vibrational (thermal) energy in the system to create sufficient free-volume to permit sequences of 6-10 main-chain carbons to move together as a unit. At this point, the mechanical behavior of the polymer changes from rigid and brittle to tough and leathery.

The “melting temperature” (Tm) is the temperature at which a material changes from solid to liquid state. In polymers, Tm is the peak temperature at which a semicrystalline phase melts into an amorphous state. Such a melting process usually takes place within a relative narrow range (<20° C.), thus it is acceptable to report Tm as a single value.

“Modulus” may be defined as the ratio of a component of stress or force per unit area applied to a material divided by the strain along an axis of applied force that result from the applied force. For example, a material has both a tensile and a compressive modulus.

“Toughness”, or “fracture toughness” is the amount of energy absorbed prior to fracture, or equivalently, the amount of work required to fracture a material. One measure of toughness is the area under a stress-strain curve from zero strain to the strain at fracture. The stress is proportional to the tensile force on the material and the strain is proportional to its length. The area under the curve then is proportional to the integral of the force over the distance the polymer stretches before breaking. This integral is the work (energy) required to break the sample. The toughness is a measure of the energy a sample can absorb before it breaks. There is a difference between toughness and strength. A material that is strong, but not tough is said to be brittle. Brittle materials are strong, but cannot deform very much before breaking.

The “degree of crystallinity” may be expressed in terms of, w_{c} (mass fraction), φ_{c} (volume fraction) and refers to mass fraction or volume fraction of crystalline phase in a sample of polymer. The mass-fraction and the volume-fraction degrees of crystallinity are related by the equation, w_{c}=φ_{c}ρ/ρ_{c}, where ρ and ρ_{c} are the mass concentrations (mass densities) of the entire sample and of the crystalline phase, respectively. The degree of crystallinity can be determined by several experimental techniques. Among the most commonly used are: (i) x-ray diffraction, (ii) calorimetry (DSC), (iii) mass density measurements, (iv) infrared spectroscopy (IR), (v) solid-state NMR spectroscopy, and (vi) vapor permeability. Unless stated otherwise, throughout this description a degree of crystallinity given for a polymer is expressed as a percentage (%) of crystallinity and expressed as a mass or volume fraction. Unless stated otherwise throughout this description a degree of crystallinity given for a polymer composition is expressed as a percentage (%) of crystallinity and expressed as a mass fraction. Measurements of crystallinity may also be determined from a modified method of differential scanning calorimetry (DSC), e.g., over a temperature range of 0 Deg. C. to 200 Deg. C., with modulation amplitude of 0.5° C. and heat rate of 6° C./minute and duration of 1 minute.

The above description of illustrated embodiments of the invention, including what is described in the Abstract, is not intended to be exhaustive or to limit the invention to the precise forms disclosed. While specific embodiments of, and examples for, the invention are described herein for illustrative purposes, various modifications are possible within the scope of the invention, as those skilled in the relevant art will recognize.

What is claimed is:

1. A stent comprising:
a scaffold comprising a poly(D,L-lactide)(PDLLA)-based polymer having at least 50% L-enantiomer and at least 4% D-enantiomer, wherein the polymer is amorphous; and
a therapeutic coating disposed over at least a portion of a surface of the scaffold, wherein the therapeutic layer comprises a drug mixed within a coating polymer composed of a poly(D,L-lactide) or poly(D,L-lactide-co-caprolactone).

2. The stent of claim 1, wherein the PDLLA-based polymer is selected from the group consisting of 50/50 PDLLA, 96/4 PDLLA, and a copolymer thereof.

3. The stent of claim 1, wherein the drug is selected from the group consisting of everolimus, rapamycin, novolimus, zotarolimus, and biolimus.

4. The stent of claim 1, wherein the coating is disposed over at least a portion of the abluminal surface of the scaffold only.

5. The stent of claim 1, wherein a lactide monomer content of the scaffold is 0.01 to 1 wt % of the scaffold.

6. The stent of claim 1, wherein a thickness of the coating is between 1 and 10 microns.

7. The stent of claim 1, wherein a thickness of the coating is between 10 and 20 microns.

8. A stent comprising:
a scaffold including a first poly(D,L-lactide)(PDLLA)-based polymer;
a primer layer on a surface of the scaffold, wherein the primer layer comprises a second PDLLA-based polymer and the primer layer is free of a therapeutic agent and;
a therapeutic layer over the primer layer, wherein the therapeutic layer comprises a third PDLLA-based polymer and a drug,
wherein the primer layer improves adhesion of the therapeutic layer to the scaffold.

9. The stent of claim 8, wherein a luminal surface of the scaffold is free of the therapeutic layer and the therapeutic layer is over an entire abluminal surface of the scaffold and part of the sidewall surfaces, wherein the primer layer is between the therapeutic layer and the scaffold surface.

10. The stent of claim 8, wherein the therapeutic layer is over at least a portion of the abluminal surface of the scaffold only, the primer layer is between the therapeutic layer and the scaffold surface, and the sidewalls and luminal surface are free of the therapeutic layer.

11. The stent of claim 8, wherein the first PDLLA-based polymer is 96/4 PDLLA or 50/50 PDLLA, the second PDLLA-based polymer is poly(D,L-lactide-co-caprolactone), and the third PDLLA-based polymer is 50/50 PDLLA.

12. The stent of claim 8, wherein the first PDLLA-based polymer is 50/50 PDLLA or 96/4 PDLLA, the second PDLLA-based polymer is poly(D,L-lactide-co-caprolactone), and the third PDLLA-based polymer is 50/50 PDLLA.

13. The stent of claim 8, wherein the first PDLLA-based polymer is 50/50 PDLLA or 96/4 PDLLA, the second PDLLA-based polymer is 50/50 PDLLA, and the third PDLLA-based polymer is 50/50 PDLLA.

14. The stent of claim 8, wherein the first PDLLA-based polymer is 50/50 PDLLA or 96/4 PDLLA, the second PDLLA-based polymer is poly(D,L-lactide-co-caprolactone), and the third PDLLA-based polymer is poly(D,L-lactide-co-caprolactone).

15. The stent of claim 8, wherein the drug is selected from the group consisting of everolimus, rapamycin, novolimus, zotarolimus, and biolimus.

16. The stent of claim 8, wherein a thickness of the primer layer is 0.2 to 2 microns; and wherein a thickness of the therapeutic layer is 2 to 20 microns.

17. The stent of claim 8, wherein the first PDLLA-based polymer is amorphous.

18. A method of coating a stent comprising:
providing a scaffold including a poly(D,L-lactide)(PDLLA)-based polymer having at least 50% L-enantiomer and at least 4% D-enantiomer, wherein the polymer is amorphous;
applying a coating composition to a surface of the scaffold, wherein the coating composition comprises a drug and a coating polymer composed of a PDLLA-based polymer dissolved in a fluid, wherein the fluid is a blend of a good solvent for the coating polymer and a poor solvent for the coating polymer; and
removing the solvent from the applied coating composition.

19. The method of claim 18, wherein a ratio of the good solvent to the poor solvent is 90/10 to 10/90 by weight.

20. The method of claim 18, wherein the good solvent is selected from the group consisting of acetone, methylene chloride, chloroform, 2-butanone, ethyl acetate, methyl acetate, tetrahydrofuran, dioxane, nitropropane, cyclohexanone, butyl benzoate, dimethylformamide, dimethylacetamide, benzyl benzoate, and N-methylpyrrolidone.

21. The method of claim 18, wherein the poor solvent is selected from the group consisting of pentane, hexane, heptane, cyclopentane, cyclohexane, methanol, ethanol, isopropanol, n-butyl acetate, diisopropyl ketone, and toluene.

22. The method of claim 18, wherein the poor solvent has a lower boiling point than the good solvent.

23. The method of claim 18, wherein the poor solvent has a boiling point lower than 55 deg C. and the good solvent has a boiling point greater than 55 deg C.

24. The method of claim 18, further comprising repeating the applying and removing steps one or more times.

25. The method of claim 18, wherein the drug is selected from the group consisting of everolimus, rapamycin, novolimus, zotarolimus, and biolimus.

26. The method of claim 18, wherein the coating is disposed over at least a portion of the abluminal surface of the scaffold only.

27. The method of claim 18, wherein a thickness of the coating is between 1 and 5 microns.

28. A method of coating a stent comprising:
providing a scaffold including a scaffold polymer composed of a poly(D,L-lactide)-based polymer having a glass transition temperature (Tg) greater than 37 deg C.;
forming a drug coating over at least a portion of the scaffold surface using a coating process, wherein the drug coating comprises a poly(DL-lactide)-based polymer, a drug, and residual solvent from the coating process, and wherein the coated scaffold is at a diameter; and
thermally processing the coated scaffold to remove the residual solvent, wherein the thermal processing comprises increasing a temperature of the coated scaffold to a temperature below the Tg of the scaffold polymer followed by reducing the temperature, wherein the thermal processing accelerates physical aging and stabilizes the dimensions of the scaffold, the density of the scaffold polymer, mechanical properties of the scaffold polymer, scaffold properties, or any combination thereof.

29. The method of claim 28, wherein a thickness of the drug coating is greater than 10 microns.

30. The method of claim 28, wherein the scaffold is at a diameter greater than a targeted deployment diameter during the thermal processing.

31. The method of claim 28, wherein the scaffold is amorphous.

32. The method of claim 28, wherein the scaffold polymer is 94/4 PDLLA or 50/50 PDLLA.

33. The method of claim 28, wherein the coating polymer is 50/50 PDLLA or poly(D,L-lactide-co-caprolactone).

34. The method of claim 28, wherein the thermal processing reduces residual solvent composition of the coating from greater than 5 wt % to less than 2 wt %.

35. The method of claim 28, wherein a temperature of the thermal processing is Tg-15 deg C. to Tg.

36. The method of claim 28, wherein the thermal processing increases the modulus of the scaffold polymer, the radial strength of the scaffold, or both.

37. A method of coating a stent comprising:
providing a scaffold including a scaffold polymer composed of a poly(D,L-lactide)-based polymer having a glass transition temperature (Tg) greater than 37 deg C.;
forming a drug coating over at least a portion of the scaffold surface using a coating process, wherein the drug coating comprises a poly(D,L-lactide)-based polymer, a drug, and residual solvent from the coating process; and
thermally processing the coated scaffold to remove the residual solvent, wherein the thermal processing comprises increasing a temperature of the coated scaffold to a temperature above the Tg of the scaffold polymer followed by reducing the temperature, wherein the thermal processing reverses physical aging of the scaffold polymer.

38. The method of claim 37, wherein a thickness of the drug coating is greater than 10 microns.

39. The method of claim 37, wherein the scaffold is at a diameter greater than a targeted deployment diameter.

40. The method of claim 37, wherein the scaffold is amorphous.

41. The method of claim 37, wherein the scaffold polymer is 94/4 PDLLA or 50/50 PDLLA.

42. The method of claim 37, wherein the coating polymer is 50/50 PDLLA or poly(D,L-lactide-co-caprolactone).

43. The method of claim 37, wherein the thermal processing reduces residual solvent composition of the coating from greater than 5 wt % to less than 2 wt %.

44. The method of claim 37, wherein a temperature of the thermal processing is Tg-15 deg C. to Tg.

45. The method of claim 37, wherein the thermal processing decreases the modulus of the scaffold polymer, increase the elongation to break of the scaffold polymer, increases the fracture resistance of the scaffold polymer, or any combination thereof.

46. A method of treating restenosis in a patient in need thereof, comprising:
implanting a bioresorbable stent comprising a scaffold and an antiproliferative drug at a stenotic section of a vessel of a patient; and
releasing the antiproliferative drug from the stent, wherein release of the drug is completed or substantially completed prior to the onset of positive remodeling of the section of the vessel.

47. The method of claim 46, wherein the release is 100% completed prior to the onset of positive remodeling.

48. The method of claim 46, wherein the release of the drug is completed or substantially completed by 2 months or 3 months after deployment of the stent.

49. The method of claim 46, wherein the release of the drug is completed or substantially completed when a radial strength of the stent is less than 350 mm Hg, or when stent radial strength is 50% of the stent's radial strength directly after deployment.

50. The method of claim 46, wherein the release of the drug is completed or substantially completed when a number average molecular weight of the scaffold is less than 47 kDa.

51. The method of claim 46, wherein the release of the drug is completed or substantially completed when scaffold's Mn is 50% of the stent's Mn directly after deployment.

52. A method of treating restenosis in a patient in need thereof, comprising:
implanting a bioresorbable stent comprising a scaffold and an antiproliferative drug at a stenotic section of a vessel of a patient;
inhibiting or preventing release of the antiproliferative drug until after early positive remodeling of the section of the vessel is completed; and
releasing the drug from the stent after early positive remodeling of the section of the vessel is completed.

53. The method of claim 52, wherein the early positive remodeling is completed when the scaffold is broken up sufficiently to allow freedom of movement of the vessel.

54. The method of claim 52, wherein the drug is released no earlier than 3 months post-implantation.

55. The method of claim 52, wherein the drug is released when the number average molecular weight of the scaffold is less than 47 kDa.

56. The method of claim 52, wherein the drug is released when scaffold's Mn is 50% of the stent's Mn directly after deployment.

57. A stent comprising:
a scaffold comprising a poly(D,L-lactide)(PDLLA)-based polymer having at least 50% L-enantiomer and at least 4% D-enantiomer, wherein the polymer is amorphous;
a therapeutic coating disposed over at least a portion of a surface of the scaffold, wherein the therapeutic layer comprises an antiproliferative drug mixed within a coating polymer composed of poly(D,L-lactide) or poly(D,L-lactide-co-caprolactone); and
a barrier coating comprising a bioabsorbable polymer over the therapeutic coating to prevent release of the drug from the stent until after early positive remodeling of the section of the vessel is completed.

58. The stent of claim 57, wherein the barrier coating is drug-free.

59. The stent of claim 57, wherein the barrier coating is tuned to degrade and allow release of the drug after 3 months.

60. The stent of claim 57, wherein the coating polymer is selected from the group consisting of poly(D,L-lactide), poly(L-lactide), polyglycolide, polycaprolactone, polydioxanone, poly(4-hydroxybutyrate), and copolymers and blends thereof.

61. The stent of claim 57, wherein the coating polymer is selected from the group consisting of aliphatic polyanhydrides, hydrophobic aromatic polyanhydrides, polyester amides, poly(ortho esters), and polyketals.
